(19) United States
(12) Patent Application Publication
(10) Pub. No.: US 2013/0035630 A1
(43) Pub. Date: Feb. 7, 2013

(54) Aptamer for the Capture, Diagnosis, Enumeration, and Eradication of Circulating Tumor Cells

(71) Applicant: Hui Chen, Crown Point, IN (US)

(72) Inventor: Hui Chen, Crown Point, IN (US)

(73) Assignee: IVDiagnostics, LLC, Crown Point, IN (US)

(21) Appl. No.: 13/568,011

(22) Filed: Aug. 6, 2012

Related U.S. Application Data
(60) Provisional application No. 61/515,246, filed on Aug. 4, 2011.

(51) Int. Cl. A61M 37/00; C12Q 1/68

(52) U.S. Cl. 604/20; 435/6.14

(57) ABSTRACT

Aptamers for use in the capture, diagnosis, enumeration and eradication of circulating tumor cells; an aptamer immobilized microfluidic chips for use in a rapid and non-invasive method of testing for cancer by capturing and detecting circulating tumor cells through the use of the aptamers immobilized microfluidic chips, and screening high risk patients for cancer thereby allowing the disease to be identified at the early stage where the prognosis of survival is the highest; and a non-invasive method of treating cancer by capturing and eradicating circulating tumor cells through the use of aptamer-photosensitizer conjugates injected into a patient's bloodstream and excited by a laser.

This application claims priority to U.S. Provisional Patent Application No. 61/515,246, filed on Aug. 4, 2011, which application is incorporated herein by reference in its entirety.

### BACKGROUND OF THE INVENTION

1.Field of the Invention

The invention relates generally to a rapid and non-invasive method of testing for cancer by detecting circulating tumor cells, and screening high risk patients for cancer thereby allowing the disease to be identified at an early stage where the prognosis of survival is the highest.

2. Description of the Related Art

Cancer is a life threatening disease. At the early stage, tumor cells containing genetic or molecular abnormalities, referred to as circulating tumor cells (“CTCs”), often enter biological fluids such as sputum, urine, or blood. More specifically, at an early stage tumors related to certain types of cancers such as lung, breast, and pancreatic cancer begin to shed CTCs into the blood stream and travel to distant sites. Instances of CTCs in peripheral blood of cancer patients have been documented through studies including those conducted by Pantel et al. (Nature Reviews Clinical Oncology, 2009), Pierga et al. (Clinical Cancer Research, 2008), and Cristonfanilli et al. (New England Journal of Medicine, 2004).

CTCs are rare among millions of normal cells in the blood stream. For example, in a study by Kahn et al. (Breast Cancer Res. Treat., 2004) CTCs were found to average 16 to 122 epithelial cells in 10 mL of whole venous blood taken from known breast cancer patients. Isolation of CTCs provides opportunity in several areas. For example, finding CTCs is a relevant risk factor for metastasis and, thus, indicative that a patient will have a poor prognosis in early stage cases. As another example, CTCs can serve as a marker for monitoring treatment susceptibility. In another example, CTCs can provide detailed insight into the biology of the source tumor and help in the exploration of targeted treatment strategies. Therefore, an effective and sensitive method for identifying a small amount of CTCs in bodily fluids is needed.

Current diagnostic techniques based on CTCs fall into three major categories: affinity ligands, size separation, or the combination of both. Many of the affinity ligand diagnostic techniques use non-specific CTC markers, e.g. epithelial cell adhesion molecule (“EpCAM”) and cytokeratins, for capturing CTCs, which only give modest catching efficiency and high false positive rates. For some cancers, there are no specific markers available for capturing CTCs. Thus, there is a desire to find new markers for the detection of CTCs, especially in cancers where no specific marker has been identified for CTC capture.

As described in Wilson et al. (Annu. Rev. Biochem., 1999) an aptamer is an emerging class of molecular recognition moiety. It is single stranded DNA, RNA, or a non-natural nucleic acid that can specifically bind to a variety of target molecules including proteins, organic molecules, ions, viruses, organelles, and cells. Aptamers are evolved from a large pool of random oligonucleotides by iterative rounds of binding and amplification, a process known as Systematic Evolution of Ligands by Exponential enrichment (“SELEX”). With a SELEX screening method, aptamers can be selected for ideal specificity, affinity, and pharmacokinetics.

Compared to their protein counterparts, aptamers have some unique features. Aptamers can be easily developed from in vitro selection and then chemically synthesized. The binding capability of aptamer is tunable with complementary oligonucleotides, or through the process of denaturing and renaturing. Also, aptamers are ideal for microfluidic chip fabrication because they are easy to immobilize or modify as they are chemically synthesized and very robust, and the variation between different batches of aptamers is very small compared to the variation found with antibodies produced by living systems. Aptamers are also very stable and have a long shelf life. Thus, it would be desirable to develop aptamers for use in CTC capture and diagnosis.

### SUMMARY OF INVENTION

An object of the invention is to develop multiple sensitive and specific aptamers as capture and diagnostic agents for CTCs using a CTC-based SELEX technique.

Another object of the invention is to immobilize the sensitive and specific aptamers on a microfluidic chip.

Another object of the invention is to use a specific CTC recognition aptamer immobilized microfluidic chip for the selective capture and collection of CTCs from bodily fluids.

Another object of the invention is to utilize the developed aptamer microfluidic chip for the detection of CTCs for diagnostic purposes and to monitor treatment susceptibility.

Another object of the invention is to utilize the CTC recognition aptamers together with photosensitizers to perform enumeration and eradication of CTCs within a patient's body.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 of the drawings is a flow diagram of a representative protein-based SELEX method of creating aptamers.

FIG. 2 of the drawings is a flow diagram of a representative cell-based SELEX method of creating aptamers.

FIG. 3 of the drawings is a simplified perspective view of an aptamer microfluidic chip which may be used for the detection of CTCs to monitor treatment susceptibility.

FIG. 4 of the drawings are color graphs of flow cytometry data and color confocal microscopy images reflecting the binding affinity of aptamers to target cells.

FIG. 5 of the drawings are color graphs of flow cytometry data reflecting the binding affinity of aptamers to target cells.

FIG. 6 of the drawings is microarray reflecting the specificity of the aptamers to selected cancer cells, and some non-cancer cells.

FIG. 7 is drawings is a flow diagram of the creation of a aptamer-photosensitizer conjugate.

FIG. 8 is a drawing of a representative laser system for CTC enumeration and eradication using an aptamer-photosensitizer conjugate.

### DETAILED DESCRIPTION OF THE INVENTION

While the present disclosure may be embodied in many different forms, the drawings and discussion are presented with the understanding that the present disclosure is an exemplification of the principles of one or more inventions and is not intended to limit any one of the inventions to the embodiments illustrated.

It is desired that the developed aptamers have excellent specificity for a specific CTC. It is also desired that the aptamers have high binding affinities to a specific CTC. Preferably multiple aptamers will be selected for use in a CTC capturing and detection technique, including CTC-specific aptamers, and non-specific CTC aptamers. The use of a combination of specific and non-specific cancer markers for CTC capturing greatly improves the capturing efficiency as these markers are able to complement to each other.

Specifically, for the detection of CTCs where no marker is available for CTC capture, it is preferred to develop a group of aptamers for specific recognition of certain specific and non-specific markers. As an example, for detecting lung cancer CTCs, epidermal growth factor receptor (“EGFR”), a surface protein that overexpresses in most cases of lung cancer, may be selected as lung cancer specific marker to develop aptamer ligands. Vimentin may be selected as non-specific marker, an important marker of epithelial-mesenchymal transition (“EMT”) of CTCs. In addition to EGFR and Vimentin, it is desired that a panel of aptamers generated for the specific CTC cells will be added to the aptamer portfolio. The selected aptamers should preferably bind to the specific CTCs with high affinity.

It is desired that the multiple aptamers are generated by a novel SELEX technique to recognize the whole CTCs by targeting multiple cancer specific markers at the same time. Aptamers are particularly useful for some cancer types that have no known marker available for diagnosis, such as lung cancer. Multiple aptamers may be used together to reduce false positive rate which is a main concern of most products in the market.

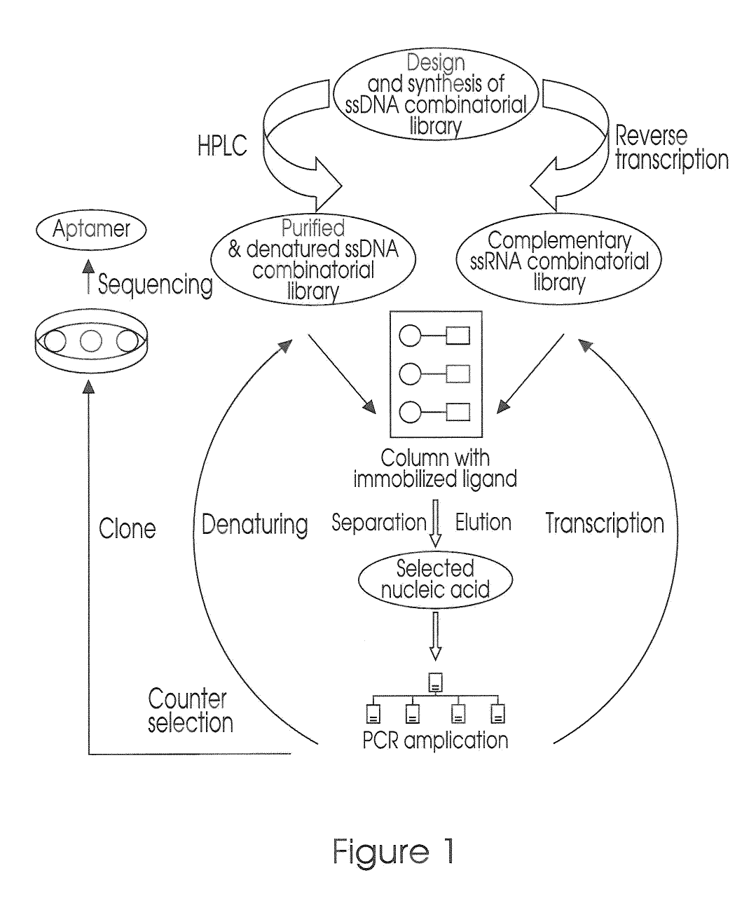

Preferably, the aptamer is selected by a single protein-based SELEX, or a cell-based SELEX. It is desired that the protein-based SELEX is used to generate aptamers for protein markers. This protein-based aptamer selection may be performed with traditional column or nitrocellulose film based technique such as the technique outlined in FIG. 1, or by high throughput commercial aptamer selection service. As reflected in FIG. 1, the protein-based SELEX may have three major steps (1) preparation of DNA/RNA library, (2) selection and screening, and (3) de-convolution and analysis of aptamers.

Preferably, the cell-based SELEX is used to develop a group of specific aptamers for CTCs. It is preferable to select aptamers using CTCs isolated from patient blood samples, which allows the better identification of representative target markers on the cell surface of CTCs. The CTC-specific aptamers are also preferably selected by using blood samples of healthy patient donors containing no CTCs for a negative selection control.

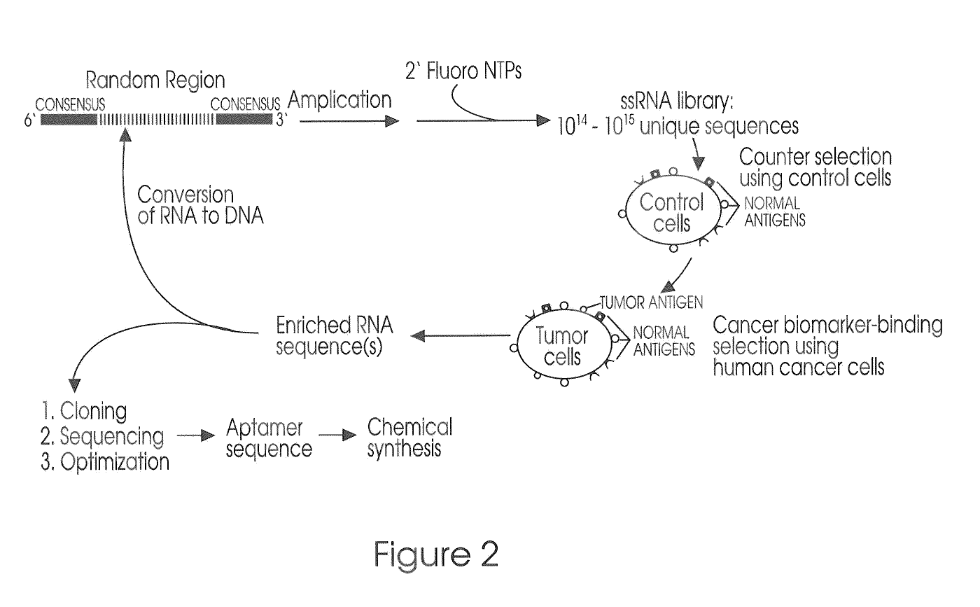

As outlined in FIG. 2, the cell-based selection strategy preferably couples selection and counter-selection steps to generate a panel of CTC-specific aptamers, which may more likely bind to CTCs with high affinity and specificity. Specifically, as outlined in FIG. 2, the cell-based SELEX may involve incubating the library with control samples for counter-selection to remove aptamers which bind to common antigens. The remaining pool may be further incubated with target CTCs. After washing with washing buffer to remove nonspecific binding aptamers and loose binding aptamers, the CTC bound aptamers may be amplified for the next round of selection, or for cloning and sequencing to identify individual aptamers in most selected pools.

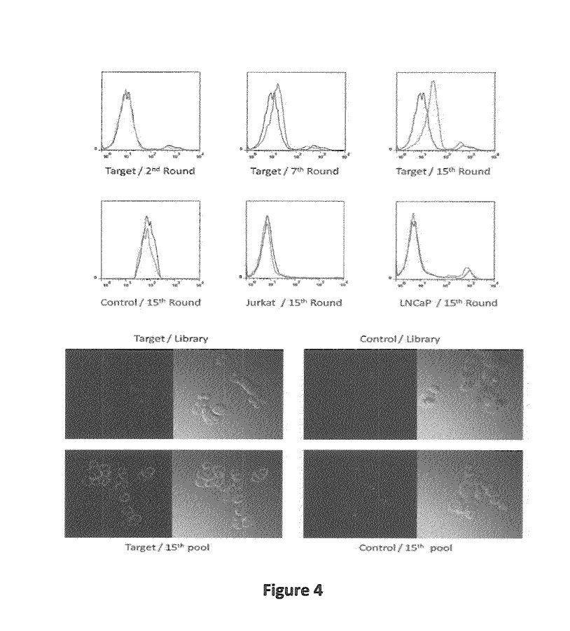

While FIG. 2 describes the cell-based SELEX technique to select the CTC-specific aptamers, FIGS. 4-6 describe ways to optimize that the cell-based SELEX technique by (1) monitoring the enrichment of the aptamers during the rounds of selection of the cell-based SELEX technique, and (2) characterizing and validating the selected aptamers which bind to target cells. Thus, FIGS. 4-6 only describe further testing conducted on the resultant CTC-specific aptamers selected by the cell-based SELEX technique.

Two techniques have been utilized to monitor the enrichment of specific cell-binding aptamers by the cell-based SELEX technique. First, flow cytometry has been used to monitor aptamer-cell binding as the numbers of rounds of selection increased during the cell-based SELEX technique. Specifically, fluorescein isothiocyanate (“FITC”)-labeled selected pools of aptamers of different rounds of selection (round 2, 7, and 15) were incubated with patient samples containing CTCs and analyzed by flow cytometry. Blood samples from healthy donors (control samples) were also incubated with FITC-labeled selected pools of aptamers and analyzed by flow cytometry. As shown in FIG. 4, as the number of selection cycles increased, a steady increase in fluorescence intensity on target cells was observed. Thus, the binding affinity of the selected pools gradually increased for target cells with increased selection cycles. However, there was no increase of fluorescence intensity, and thus, no increased binding affinity for any of the three control cells. These results indicate that the aptamers specifically recognizing surface biomarkers on CTCs are enriched with increased selection cycles.

Secondly, confocal microscopy has been used to visualize cells bound to the selected aptamers after the FITC-labeled aptamers were incubated with the target cells and control cells. As shown in FIG. 4, after the FITC-labeled 15th round pool was incubated with target cells and control cells, the fluorescent dye-labeled aptamers were bound to the surface of target cells, which is observed by the fluorescence. There was no observation of aptamer binding to control cells. Only background fluorescence was observed on cells incubated with the initial library.

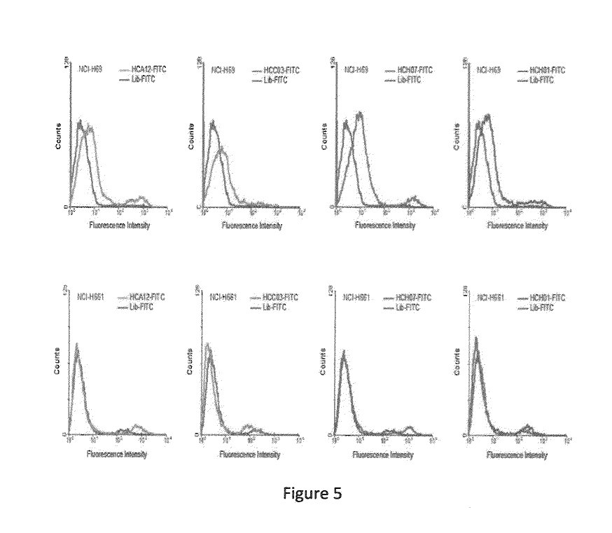

Studies have also been conducted to characterize and validate the selected aptamers bound to the target cells. After determining which rounds of selection of the cell-based SELEX technique provided the most enriched aptamer pool, that pool was used to isolate individual aptamer sequences. The most enriched pool was cloned into a plasmid vector and transformed into Escherichia coli. The plasmid DNA was then sequenced by a high-throughput sequencing method. The obtained sequences were analyzed and aligned using Sequencher 5.0 software from Gene Codes Corporation. Then, the repetitive sequences were synthesized for further characterization. As shown in FIG. 5, these aptamer sequences (HCA12, HCC03, HCH07, and HCH01) were labeled with FITC, incubated with target cells (small cancer lung cells (“SCLC”)), and tested by flow cytometry analysis. The SCLC line that was utilized was NC1-H69. The aptamers did bind to the target cells. However, as shown in FIG. 5, the labeled aptamer sequences (HCA12, HCC03, HCH07, and HCH01) that were incubated with control cells, NC1-H661, a non-small cell lung cancer cell line, did not bind to the control cells. The binding affinities of selected aptamers to the CTCs were determined to be in the nanomolar range by saturation analysis. Saturation analysis is a method to measure the relative cell surface binding affinities of the developed aptamers. The apparent dissociation constants (Kd) of these aptamers are from 25 nM to 250 nM.

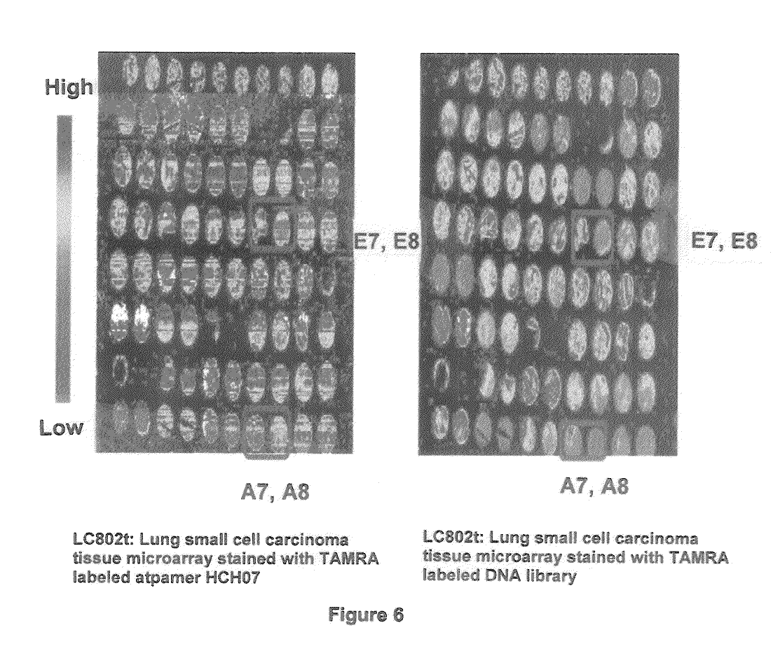

As shown in FIG. 6, these aptamers were further tested with various lung cancer cell lines by utilizing a tissue microarray made from clinical lung cancer samples to validate their specificity. Lung small cell carcinoma tissue microarrays were stained with tetramethylrhodamine (“TAMRA”) labeled aptamer (left) and TAMRA labeled DNA library (right). The stained arrays were analyzed by array scanning. The lung small cell carcinoma tissue microarrays contained 40 cases with duplicated cores per case. Among the 40 cases, 2 are tumor adjacent normal sample and 3 are non-malignant normal lung tissue (top row), 35 cases are small cell lung carcinoma (from 2nd-8th row). Most of the cores on the positive slide showed strong fluorescence except the normal lung tissue cores used as controls on the same slide (top row). In contrast, the cores on the negative slide maintained a low level of average fluorescence signal. The tissue microarray stained by dye labeled aptamers showed statistically higher fluorescence signal than the tissue microarray stained by non-specific library, indicating that selected aptamers have the ability to distinguish clinical cancer samples.

In one embodiment, after the aptamer is selected, developed and optimized, it may be immobilized on microfluidic chip. Preferably, the microfluidic chip is fabricated and optimized by using poly (dimethylsiloxane) (“PDMS”).

In order for the selected aptamers to be useful for capturing of cancer cells on microfluidic chip, they preferably are immobilized on the surface of microfluidic chip. Preferably, aptamers will be modified with biotin and incubated with avidin treated surface of microfluidic chip. Once the aptamer is immobilized through biotin-avidin interaction, it can be used to capture CTCs in blood sample. Avidin is a protein derived from both avians and amphibians that shows considerable affinity for biotin, a co-factor that plays a role in multiple eukaryotic biological processes. Avidin has the ability to bind up to four biotin molecules. The avidin-biotin complex is the strongest known non-covalent interaction (Kd=10-15M) between a protein and ligand. The bond formation between biotin and avidin is very rapid, and once formed, is unaffected by extremes of pH, temperature, organic solvents and other denaturing agents. These features of biotin and avidin are useful for immobilizing aptamers to microfluidic chip surfaces. It is contemplated that other molecules that have strong binding properties may also be utilized to immobilize aptamers to a microfluidic chip surface. It is desired that the aptamer microfluidic chip will also be optimized for ligand density, flow velocity, and incubation time.

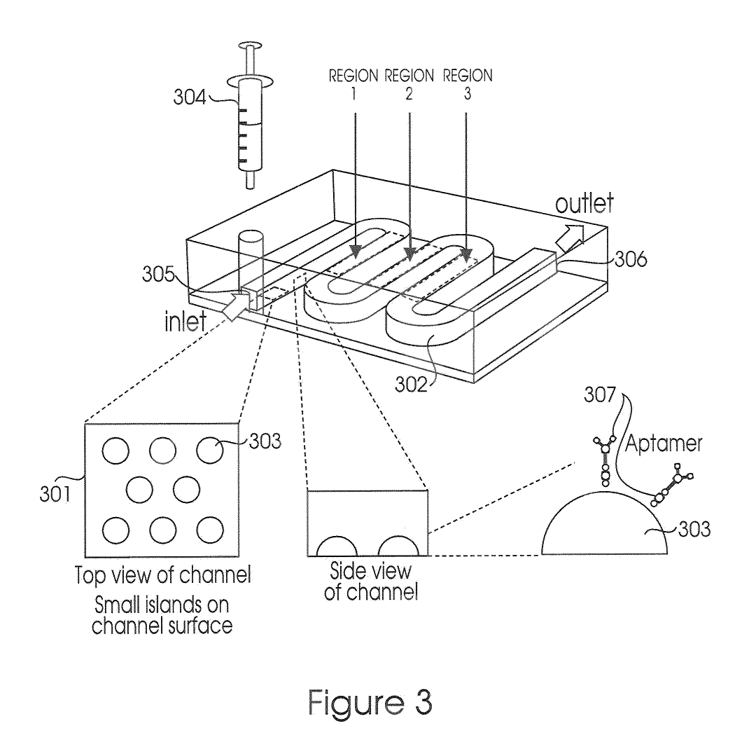

The aptamer microfluidic chip utilizes both size restriction and affinity capturing for the collection of CTCs. As shown in FIG. 3, an “islands” design (301) is used for size restriction. Uniformly fabricated “islands” (301) in microchannel (302) intrude to the sample flow, and the gaps between islands can impede the large CTC cells when small blood cells flow through without hindrance. Meanwhile these “islands” (301) dramatically increase the contact area of affinity ligands (aptamers) (303) with the sample. For affinity capturing, the immobilized aptamers (303) are on the surface of the microfluidic chip. With the help of “islands” design (301), aptamers (303) can more efficiently catch CTC cells (307) in the sample flow. Another feature of the aptamer microfluidic chip is the extended length of microchannel (302), which has two advantages. First, the longer the microchannel (302), the longer incubation time of CTC cells with affinity ligands (aptamers) (303). Second, multiple regions can be used to immobilize different affinity ligands (aptamers) (303) targeting different markers. This design allows the use of combining multiple ligands (303) for maximized catch efficiency of CTC cells. It is contemplated that the design of the aptamer microfluidic chip is not limited to the design reflected in FIG. 3; rather the microchannels (302) and the aptamers (303) in the microchannels (302) may be modified in shape and in size. For example, the microchannels may have an alternative arrangement. As another example, the inlet of the microchannel (305) or outlet of the microchannel (306) may be modified.

Briefly, as shown in FIG. 3, the capturing and detection technique preferably involves injecting a bodily fluid sample into the aptamer immobilized microfluidic chip (300) through a syringe (304) into an inlet (305). It is contemplated that the sample enters and flows through the aptamer immobilized microfluidic chip (300). While passing through the microchannel (302), the bodily fluid, possibly containing CTCs, passes over the islands (301) of aptamers (303). If a CTC is present the aptamer (303) will efficiently catch CTC cells in the sample flow. After the capture of the CTCs the collected CTCs may be quantified. Preferably, the CTCs are dye-labeled and can be detected by confocal microscopy after being captured by immobilized aptamers (303). The images of captured cells may be analyzed by software. Then the information derived can be used to provide a prognosis, or can be used to monitor treatment susceptibility. Also, it is contemplated that the captured CTCs may provide detailed insight into the biology of the tumor and allow exploration of targeted treatment strategies. For example, the isolated CTCs may be used for tumor genotyping by using allele-specific polymerase chain reaction (“PCR”) to perform EGFR and kirsten ras oncogene (“KRAS”) mutational analysis on the DNA recovered from the CTCs. Mutations in the EGFR and KRAS genes are frequently found in cancers.

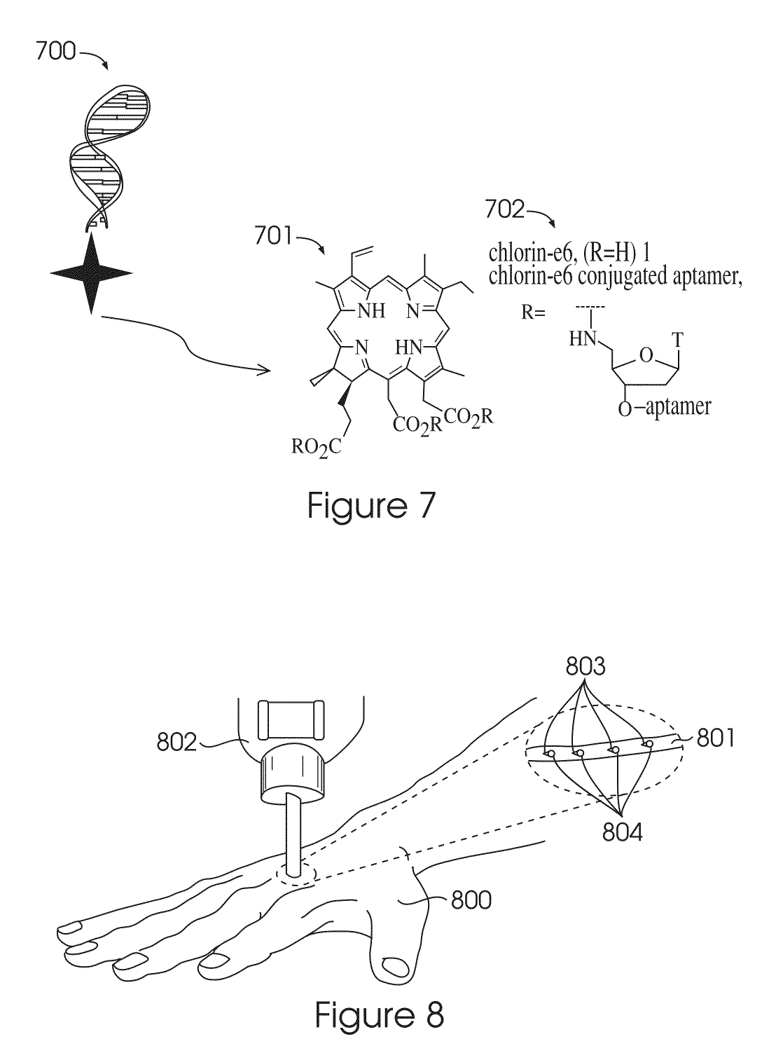

As shown in FIG. 7, the apatmers (700) developed for CTC detection may also be covalently linked to photosensitizers (701) to form a aptamer-photosensitizer conjugate (702) which may act to perform enumeration and eradication of CTCs. It is contemplated that the photosensitizer (701) is one that undergoes photochemical reactions to produce a cytotoxic agent. Preferably, the photosensitizer (701) is Chlorin-e6. Chlorin-e6 undergoes photochemical reactions upon absorption of visible light (650 nm) to produce highly-reactive singlet oxygen which is the cytotoxic agent responsible for causing irreversible tumor cell destruction.

As shown in FIG. 7, in one embodiment of the invention, the aptamers (700) are linked to the photosensitizer (701) Chlorin-e6 by chemical synthesis. The aptamer-photosensitizer conjugate (702) are synthesized using standard phosphoramidite chemistry. Preferably, the amine of the oligonucleotides are covalently appended to the free carboxyl groups of Chlorin-e6 using a coupling reagent. The aptamer-photosensitizer conjugate (702) may be purified with high-performance liquid chromatography (“HPLC”). The aptamer-photosensitizer conjugate (702) may be tested with cultured cancer cells spiked in blood and CTCs isolated from patient samples for binding and efficacy.

As shown in FIG. 8, it is contemplated that the CTCs (804) linked to the aptamer-photosensitizer conjugate (803) may be detected, enumerated, and eradicated when the fluorescence of the photosensitizer is excited by a laser system (802). It is contemplated the patient is injected with the aptamer-photosensitizer conjugate (803). While in the bloodstream the aptamer-photosensitizer conjugate (803) links to the CTCs (804). FIG. 8 depicts detection of a aptamer-photosensitizer conjugate (803) in the bloodstream (801) in a patient's hand (800). The laser system (802) preferably has integrated laser excitation and emission optics, and a software controlled CTC counting system. It is desired that the laser is emitted by a probe of fiber-optic array. An example of a preferred laser system is described in pending U.S. Application Ser. No. 13/080,544, and is hereby incorporated by reference in its entirety. When the CTCs (804) linked to the aptamer-photosensitizer conjugate (803) in the bloodstream moves past the laser above the patient's hand, the fluorescence of the photosensitizer is excited by a laser system (802). The laser system (802) acts to excite the aptamer-photosensitizer conjugate (803) which causes it to fluoresce and to also produce highly-reactive singlet oxygen which is the cytotoxic agent responsible for causing irreversible CTC destruction, and eradication of the CTC (804). The laser system also preferably scans the cells in the bloodstream to provide CTC enumeration.

It is contemplated that when every CTC (804) is tagged by the aptamer-photosensitizers conjugate (803) and detected by fluorescence on laser system (802), the aptamer-photosensitizers conjugates (803) will simultaneously produce singlet oxygen to eradicate the CTCs (804).

The foregoing description and drawings merely explain and illustrate the invention and the invention is not limited thereto. While the specification in this invention is described in relation to certain implementation or embodiments, many details are set forth for the purpose of illustration. Thus, the foregoing merely illustrates the principles of the invention. For example, the invention may have other specific forms without departing from its spirit or essential characteristic. The described arrangements are illustrative and not restrictive. To those skilled in the art, the invention is susceptible to additional implementations or embodiments and certain of these details described in this application may be varied considerably without departing from the basic principles of the invention. It will thus be appreciated that those skilled in the art will be able to devise various arrangements which, although not explicitly described or shown herein, embody the principles of the invention and, thus, within its scope and spirit. All patents, patent applications, and publications cited herein are incorporated by reference in their entirety.

What is claimed is:

1. A method of detecting cancer comprising:
injecting a bodily fluid sample into a aptamer immobilized microfluidic chip, wherein the aptamer is specific for the type of circulating tumor cell (“CTC”) desired for capture,
selectively capturing the CTC by binding to the aptamer immobilized microfluidic chip, and
analyzing the captured CTC.

2. The method of claim 1 wherein the type of cancer is selected from the group consisting of lung cancer, breast cancer, and pancreatic cancer.

3. A method of claim 1 wherein the aptamer immobilized microfluidic chip also comprises at least one non-specific aptamer.

4. A method of claim 1 wherein the bodily fluid sample is injected into an inlet on the aptamer immobilized microfluidic chip.

5. A method of claim 4, wherein the bodily fluid sample is injected into the inlet on the aptamer immobilized microfluidic chip through a syringe.

6. The method of eradicating CTCs in a cancer patient comprising:
injecting into a patient's bloodstream an aptamer-photosensitizer conjugate, wherein the aptamer is specific for the type of circulating tumor cell (“CTC”) desired for capture,
selectively capturing the CTC by binding it to the aptamer-photosensitizer conjugate, creating a CTC-aptamer-photosensitizer conjugate,
passing the CTC-aptamer-photosensitizer conjugate through the patient's bloodstream and past a laser system positioned outside the patient's body and facing towards a blood vessel of the patient's body,
emitting light from the laser system onto the patient's body at the location of the blood vessel,
exciting the CTC-aptamer-photosensitizer conjugate passing through the blood vessel at the location of the emitting light,
causing CTC destruction in the blood vessel, and
eradicating the CTC.

4. The method of detecting cancer as claimed in claim 6, wherein the type of cancer is selected from the group consisting of lung cancer, breast cancer, and pancreatic cancer.

6. A method of claim 6 wherein the laser system also enumerates the CTC-aptamer-photosensitizer conjugates passing through the blood vessel.

7. A method of claim 6, wherein the photosensitizer of the aptamer-photosensitizer conjugate undergoes photochemical reactions to produce a cytotoxic agent.

8. A method of claim 7, wherein the photosensitizer of the aptamer-photosensitizer conjugate is Chlorin-e6.
